(19) United States
(12) Patent Application Publication
(10) Pub. No.: US 2003/0144336 A1
(43) Pub. Date: Jul. 31, 2003

(54) Method of selecting nonsedating H1-antagonists

(75) Inventors: Cuiping Chen, East Lyme, CT (US); Jae S. Lee, Old Saybrook, CT (US); John W. Watson, Ledyard, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/160,149

(22) Filed: May 30, 2002

Related U.S. Application Data
(60) Provisional application No. 60/315,083, filed on Aug. 27, 2001.

(51) Int. Cl. C12Q 1/00; A61K 31/4172

(52) U.S. Cl. 514/400; 435/4; 800/3

(57) ABSTRACT

A method is described in accordance with which one may select a nonsedating histamine H₁-antagonist; comprising the step of determining whether a candidate H₁-antagonist is a substrate for P-gp, especially P-gp expressed by MDR1 or mdr1a/1b, comprising the step of determining the brain-to-plasma AUC ratio in mdr1a/1b KO mice, and in WT mice, and selecting said candidate where the brain-to-plasma AUC ratio for said KO mice to the brain-to-plasma concentration ratio for said WT mice is 1.5 or greater.

### 1.0 REFERENCE TO RELATED APPLICATIONS

This application claims priority from U.S. provisional application No. 60/315,083, filed Aug. 27, 2001.

### 2.0 BACKGROUND OF THE INVENTION

The present invention concerns a method of selecting nonsedating H₁-antagonists, and is based on the discovery that nonsedating H₁-antagonists are substrates for P-glycoprotein (P-gp), while at the same time sedating H₁-antagonists are not substrates for P-gp. P-gp is a single gene product of the multiple drug resistance gene, MDR1 in humans. In mice there are two genes, mdr1a and mdr1b, which encode two P-gp isoforms. Mouse mdr1a and mdr1b P-gps together seem to fulfill the same functions as human MDR1 P-gp. See Croop J M, Raymond M, Haber D, Devault A, Arceci R J, Gros P and Housman D E (1989), “The three mouse multidrug resistance (mdr) genes are expressed in a tissue-specific manner in normal mouse tissues,” Mol Cell Biol 9: 1346-50; and Gottesman M M and Pastan I (1993), “Biochemistry of multidrug resistance mediated by the multidrug transporter,” Annu Rev Biochem 62:385-427. P-gp uses ATP via hydrolysis to provide the energy required to pump a variety of compounds out of the cell and has many substrates of diverse structure, including anti-cancer drugs, digoxin, and verapamil.

P-gp is, accordingly, an efflux transporter system that is located at the apical domain of the epithelium of intestine and kidney, bile canaliculi, and the endothelium of the blood-brain barrier. These locations are ideal for facilitating the excretion of drugs and limiting their intracellular accumulation. See Borst P, Evers R, Kool M and Wijnholds J (1999), “The multidrug resistance protein family,” Biochimica Biophysica Acta 1461: 347-57.

Antagonists of H₁ histamine receptors, referred to herein as H₁-antagonists, are one of the key therapeutic agents currently used in the treatment of a number of allergic disorders, and inflammatory disorders having an allergic component. Such disorders particularly include, but are not limited to, rhinitis, conjunctivitis, uveitis, dermatitis, urticaria, bronchitis, and asthma.

Histamine, β-aminoethylimidazole, is a hydrophilic molecule which acts through three different receptors, H₁, H₂, and H₃. Histamine is a preformed mediator that is stored in mast cells and that is released as the result of the interaction of antigen with IgE antibodies on the mast cell surface. Histamine plays a central role in mediating hypersensitivity and allergic responses. The major symptoms of the allergic response consist of constriction of the bronchi, decrease in blood pressure, increased capillary permeability, and edema formation. Histamine contracts many smooth muscles, e.g., those of the bronchi, while also being able to cause powerful relaxation of other smooth muscles, e.g., those of small blood vessels. Bronchoconstriction is mediated by H₁ receptors, as is the hypotensive response and the formation of edema. Stimulation of sensory nerve endings is another effect attributable to mediation by H₁ receptors.

Histamine functions as a neurotransmitter in the central nervous system (CNS), and H₁ receptors are found throughout the CNS and are densely concentrated in the hypothalamus. Histamine also increases wakefulness through mediation by the H₁ receptors, which explains the tendency of the classical H₁-antagonists “antihistamines” to cause sedation.

H₁-antagonists inhibit most responses of smooth muscle to histamine, including vasoconstrictor effects, and the more rapid vasodilator effects. H₁ antagonists strongly block the action of histamine that results in increased capillary permeability and formation of edema and wheal.

There have been two generations of H₁-antagonists in the course of the discovery and commercialization of such therapeutic agents. The first generation H₁-antagonists, typified by such agents as diphenhydramine, sold as BENADRYL; triprolidine, sold as ACTIFED, and hydroxyzine, sold as ATARAX, have been found to produce histamine blockade at H₁-receptors in the CNS, but they can both stimulate and depress the CNS. Where stimulation occurs, patients become restless, nervous, and unable to sleep. Depression of the CNS, however, is far more common, and manifests itself by diminished alertness, slowed reaction times, and somnolence. Therefore, first generation H₁-antagonists are also referred to as sedating H₁-antagonists.

Second generation H₁-antagonists such as cetirizine, sold as ZYRTEC; loratadine, sold as CLARITIN and desloratadine, the active metabolite of loratadine; terfenadine, sold as SELDANE; and fexofenadine, the active metabolite of terfenadine, sold as ALLEGRA; are nonsedating and thus represent an advance in therapeutics. See Kay G G and Harris A G (1999), “Loratadine: a nonsedating antihistamine. Review of its effects on cognition, psychomotor performance, mood and sedation,” Clinical Exp Allergy 29 (Suppl 3): 147-50.

When sedation is objectively measured in patients being treated with a second generation H₁-antagonist, using sleep latency, EEG, standardized performance tests, and other procedures, the results are similar to those obtained with placebo. Consequently, second generation H₁-antagonists are frequently referred to as nonsedating antihistamines. This improvement in toleration profile with respect to impaired psychomotor function, including sedation or somnolence, results from reduced CNS penetration, as already indicated. See Yanai K, Okamura N, Tagawa M, Itoh M, and Watanabe T (1999), “New findings in pharmacological effects induced by antihistamines: from PET studies to knock-out mice.” Clinic Exp Allergy 29: 29-36.

The underlying mechanism by which the penetration of brain tissue by second generation H₁-antagonists is limited has yet to be fully understood and defined by the art. The potential influence of the P-gp efflux transporter system has been postulated, because P-gp is found in the apical membrane of endothelial cells in the brain. P-gp operates in the direction of efflux from brain to blood, i.e., it decreases the apparent permeability of the blood-brain barrier to substrates of P-gp, thereby reducing CNS effects. See Yokogawa K, Takahashi M, Tamai I, Konishi H, Nomura M, Moritani S, Miyarnoto K and Tsuji A (1999), “P-glycoprotein-dependent disposition kinetics of tacrolimus: studies in mdr1a knock-out mice,” Pharm Res 16:1213-8; Cvetkovic M, Leake B, Fromm M F, Wilkinson G R and Kim R B (1999), “OATP and P-glycoprotein transporters mediate the cellular uptake and excretion of fexofenadine,” Drug Met Disp 27:866-71; and Chen C and Pollack G M (1999), “Altered disposition and anti-nociception of [D-Penicillamine] enkephalin in mdr1a-gene-deficient mice,” J Pharmacol Exp Ther 287: 545-552.

The creation of P-gp knock-out (KO) mice has greatly enhanced the ability of biologists and medicinal chemists to study the effect of P-gp on the blood-brain disposition of xenobiotics. See Schinkel A H (1998) “Pharmacological insights from P-glycoprotein knock-out mice,” International J Clinical Pharmacol Ther 36: 9-13. The term “knock-out” as used herein is intended to refer to partial or complete suppression of the expression of at least a portion of a protein encoded by an endogenous DNA sequence in a cell. Three types of P-gp KO mice have been generated in the art. See Schinkel A H, Smit J J, van Tellingen O, Beijnen J H, Wagenaar E, van Deemter L, Mol C A, van. der Valk M A, Robanus-Maandag E C, te Riele H P J, Berns A J M and Borst P (1994), “Disruption of the mouse mdr1a P-glycoprotein gene leads to a deficiency in the blood-brain barrier and to increased sensitivity to drugs,” Cell 77: 491-502; Schinkel A H, Mayer U, Wagenaar E, Mol C A, van Deemter L, Smit J J, van der Valk M A, Voordouw A C, Spits H, van Tellingen O, Zijlmans J M, Fibbe W E and Borst P (1997), “Normal viability and altered pharmacokinetics in mice lacking mdr1-type (drug-transporting) P-glycoproteins,” Proc Natl Acad Sci USA 94: 4028-33; and Croop et al., Ibid. The KO mice that have been created by molecular biologists in the art are commonly identified as mdr1a KO mice, mdr1b KO mice, and mdr1a/1b double KO mice. Mouse mdr1a is located predominantly in the intestinal epithelium, the brain endothelium, and the testis; whereas, mouse mdr1b is located predominantly in the adrenal gland, the pregnant uterus, and the ovaries. In addition, both genes are substantially expressed in many other tissues, including liver, kidney, lung, heart and spleen. See Croop et al., ibid.; and Schinkel et al. (1994), ibid.

For compounds that are substrates for the drug transporter P-gp, the brain-to-blood concentration ratio at steady state, or brain-to-plasma area-under-the-curve (AUC) ratio after a same dose of administration, is significantly higher in P-gp KO mice compared to the genetically competent, i.e., “wild type” (WT) counterpart. See Chen and Pollack, ibid. The majority of studies conducted to examine the impact of P-gp on the disposition of drugs and other compounds has relied on mdr1a single KO mice. See Chen and Pollack, ibid.; and Kusuhara H, Suzuki H, Terasaki T, Kakee A, Lemaire M and Sugiyama Y (1997), “P-glycoprotein mediates the efflux of quinidine across the blood-brain barrier,” J Pharmacol Exp Ther 283: 574-80. However, over-expression of mdr1b in these mdr1a KO mice results in increased biliary excretion of P-gp substrates. Mouse mdr1b also exists in liver and kidney tissues, which will significantly reduce the amount of P-gp substrates available for brain penetration. See Schinkel et al. (1994), ibid. In contrast, a preferred embodiment of the present invention uses mdr1a/1b double KO mice to obtain the data that defines the impact of P-gp on the disposition of potential P-gp substrates in vivo. In said mdr1a/1b double KO mice, all expression of P-gp has been eliminated.

In accordance with the present invention, it has been discovered that there is a link, i.e., a connecting factor, between non-sedation of second generation H₁-antagonists and the drug transporter P-gp. The art has heretofore shown that terfenadine is an inhibitor of and a substrate for P-gp in vitro. See Hait W N, Gesmonde J F, Murren J R, Yang J M, Chen H X and Reiss M (1993), “Terfenadine (Seldane): a new drug for restoring sensitivity to multidrug resistant cancer cells,” Biochemical Pharmacol 45: 401-6; and Raeissi S D, Hidalgo I J, Segura-Aguilar J, Artursson P (1999), “Interplay between CYP3A-mediated metabolism and polarized efflux of terfenadine and its metabolites in intestinal epithelial Caco-2 (TC7) cell monolayers,” Pharm Res 16: 625-32. Two separate studies have also shown that fexofenadine is a P-gp substrate. See Cvetkovic et al., ibid.; and Soldner A, Christians U, Susanto M, Wacher V J, Silverman J A and Benet L Z (1999), “Grapefruit juice activates P-gp-mediated drug transport,” Pharm Res 16: 478-85. These studies suggest the presence of a P-gp mediated efflux of terfenadine and fexofenadine, which is consistent with, although it does not suggest, a link between non-sedation of second generation H₁-antagonists and the efflux pump functioning of P-gp, upon which the present invention is based. These and other aspects of the present invention are demonstrated further below.

### 3.0 SUMMARY OF THE INVENTION

The present invention relates to a method for selecting a nonsedating H₁-antagonist comprising determining whether a candidate H₁-antagonist is a substrate for P-gp. The present invention especially relates to such a method where the P-gp is expressed by MDR1 or by mdr1a/1b. In particular, the present invention relates to a method for selecting a nonsedating H₁-antagonist by ascertaining whether a candidate H₁-antagonist is a substrate for P-gp, comprising the step of determining the brain-to-plasma concentration ratio of said candidate in mdr1a and mdr1a/1b KO mice, and in WT mice, and selecting said candidate where the brain-to-plasma concentration ratio for said KO mice to the brain-to-plasma concentration ratio for said WT mice is 1.5 or greater, preferably 3.0 or greater, more preferably 5.0 or greater, more preferably still 10.0 or greater, and most preferably 15.0 or greater. More in particular, the present invention is also concerned with the above-described method for selecting a nonsedating H₁-antagonist wherein said selection is based on the ratio AUC values for brain and plasma measured in said KO and WT mice.

The present invention is further concerned with a method for selecting a non-sedating sedating H₁-antagonist by ascertaining whether an H₁-antagonist candidate is a substrate for P-gp comprising (1) the step of making a determination that is carried out in vitro in a P-gp over-expressed cell line vs. the appropriate control cell line; (2) the step of making a determination that is carried out in situ in a P-gp competent animal in the presence vs. the absence of a P-gp modulator, or that is carried out in situ in a P-gp KO or compromised animal vs. its P-gp competent counterpart; or (3) the step of making a determination that is carried out in vivo in a P-gp competent animal in the presence vs. the absence of a P-gp modulator, or that is carried out in vivo in a P-gp KO or compromised animal vs. its P-gp competent counterpart.

The present invention in particular concerns the above-described method where said P-gp is over-expressed by MDR1 or mdr1a or mdr1a/1b in vitro by a cell line, and more particularly where said cell line comprises, but is not limited to, Chinese Hamster Ovary (CHO)cells, Caco-2 cells, Madin-Darby Canine Kidney (MDCK) cells, KB 8-5 cells, NIH 3T3 cells, or LLC-PK1 cells. See Martin C, Berridge G, Higgins C F, Mistry P, Charlton P, and Callaghan R (2000), “Communication between multiple drug binding sites on P-gp,” Mol Pharmacol 58: 624-32. The determination of whether a candidate H₁-antagonist is a P-gp substrate is based on the ratio of permeability from basal-to-apical vs. apical-to-basal calculated from the results obtained using a P-gp overexpressed cell line and the appropriate control cell line. The present invention also in particular concerns a method of determining whether an H₁-antagonist candidate is a substrate for P-gp using in situ organ perfusion, e.g., using intestine or brain perfusion, wherein said determination is based on the difference in uptake of said candidate H₁-antagonist calculated from the results obtained using an organ of a P-gp competent animal in the presence vs. the absence of a P-gp modulator; or said determination is based on uptake of said H₁-antagonist in an organ of a P-gp KO or compromised animal vs. its P-gp competent counterpart.

The present invention further in particular concerns a method of determining whether an H₁-antagonist candidate is a substrate for P-gp in vivo in an animal, where said animal includes, but is not limited to, a mouse, a rat, a dog, or a monkey. Said determination is based on calculations obtained by comparing PK values in a P-gp competent animal in the presence vs. the absence of a P-gp modulator; or calculations obtained by comparing PK values in P-gp KO or compromised animals to PK values from their P-gp competent counterparts

### 4.0 DETAILED DESCRIPTION OF THE INVENTION

The fact that first generation H₁-antagonists are sedating, while second generation H₁-antagonists are non-sedating, or at least less sedating, has long been known; but the precise mechanism that accounts for this important difference in pharmacological properties has not been determined. It has been assumed that sedation is a biological activity emanating from, or controlled by the brain; however, there are numerous factors known to affect the disposition of a given therapeutic agent in brain tissue. Factors such as physical-chemical properties, e.g., molecular weight (Mr), logD, and ionization constant; as well as the presence of active transporter systems, both uptake and efflux, at the blood-brain barrier may determine the blood-brain translocation of xenobiotics. Differences in physical-chemical properties may thus play a role in determining the extent of brain penetration and CNS exposure to said H₁-antagonists.

For example, sedating H₁-antagonists in general have smaller molecular weights, i.e., <350, compared to the molecular weights of nonsedating H₁-antagonists, i.e., 340-502. However, Mr differences alone cannot provide an explanation for the observed differences in brain penetration between sedating and nonsedating H₁-antagonists. For example, the data set forth herein shows that desloratadine, the active metabolite of loratadine, has a Mr of 338.9, which is comparable to that of hydroxyzine, which is 347.9; and yet brain tissue levels of desloratadine are undetectable, while the level of hydroxyzine observed in brain tissue of WT mice two minutes after the same i.v. dose, is ˜8 μg/mL. With regard to active transporter systems, few studies have been conducted to examine whether active transporters are responsible for the difference in blood-brain translocation of sedating vs. non-sedating H₁-antagonists in any species, including humans.

There are a number of blood-brain barrier active transporter systems, both uptake and efflux, that are known in the art. One such system comprises P-gp transporter. P-gp has been shown to decrease brain penetration of a variety of compounds with diverse structures, Schinkel et al. (1994), ibid; Chen and Pollack (1999), ibid.

There has been speculation in the art concerning a possible relationship between H₁-antagonists and P-gp. See Timmerman H (1999), “Why are non-sedating antihistamines non-sedating?” Clinical & Exp Allergy 29 (Suppl 3): 13-8; Timmerman H (2000), “Factors involved in the absence of sedative effects by the second-generation antihistamines,” Allergy: Eur J Allergy & Clinical Immunl 55 (Suppl 60): 5-10; and Renwick, ibid. The likelihood of interactions occurring between H₁-antagonists and P-gp has been suggested, because of the structural similarities between H₁-antagonists and Ca⁺⁺ channel blockers, since some Ca⁺⁺ channel blockers have been found to be P-gp substrates or inhibitors, Timmerman (2000), ibid. Accordingly, terfenadine, originally described as a Ca⁺⁺ channel blocker, has been found to be a P-gp substrate. See Raeissi (1999), ibid. Fexofenadine, another non-sedating H₁-antagonist, has been shown to be a P-gp substrate both in an MDR1 transfected system and in mdr1a KO mice; Cvetkovic et al., ibid.

In accordance with the present invention, an investigation was carried out in mdr1a/1b KO mice and WT mice after s.c. (subcutaneous) administration of cetirizine (20 mg/kg). A 2- to 5-fold higher brain concentration of cetirizine was observed in KO vs. WT mice, while the plasma concentrations were comparable, thereby suggesting that cetirizine is a P-gp substrate. It is noted that the brain-to-plasma ratios change with time. In this investigation, the ratio increased with time until it reached a maximum at 2 hours. As a result, difficulties would be encountered in attempting to use a brain-to-plasma concentration ratio at a single randomized time point for the purpose of comparison screening a series of compounds for their P-gp substrate affinities. Instead, the AUC ratio between brain and plasma for both strains of mice was used to assess the extent of brain penetration for all of the compounds tested in the investigations described herein.

It has been found that, in general, the range of values for the brain-to-plasma AUC ratios are much higher for sedating H₁-antagonists, i.e., from 3.8 to 9.0, than for non-sedating H₁-antagonists, i.e., from 0.02 to 1.65, in WT mice, where P-gp exists in the brain endothelium. This is deemed to be a demonstration of the ability of P-gp to act as an efflux pump that expels a substrate in the form of a nonsedating H₁-antagonist from brain tissue to blood. In the absence of the mdr1a/1b gene, the partitioning of such P-gp substrates as loratadine, cetirizine, and desloratadine to brain tissue is no longer restricted by P-gp, but instead is governed by the physico-chemical properties of said substrates. The increase in magnitude of brain partitioning of said substrates in KO mice compared to WT mice demonstrates the high level of their affinity as P-gp substrates.

In accordance with the present invention, it has been concluded that all non-sedating H₁-antagonists are substrates for P-gp, based on the testing of numerous such agents, described further herein, that has determined that they are P-gp substrates. A 1.6-, 4.4-, and >14-fold higher brain-to-plasma AUC ratio was observed in KO mice compared to WT mice for loratadine, cetirizine, and desloratadine, respectively. Based on the test results of the present study, the above-recited ratios suggest that loratadine has the lowest, while desloratadine has the highest P-gp substrate affinity. On the other hand, it has been observed that there is no significant difference in systemic distribution between KO and WT mice for cetirizine, loratadine, and desloratadine. From this it has been concluded that penetration of brain tissue does not affect the systemic clearance of the above-mentioned nonsedating H₁-antagonists. This conclusion is consistent with results obtained using other P-gp substrates; Chen and Pollack, ibid.

In accordance with the present invention, the mammalian species which is the source of P-gp is not a critical factor in determining whether an H₁-antagonist candidate is a substrate for P-gp in general. Any such P-gp may be the basis upon which it is determined whether or not an H₁-antagonist candidate is a substrate for said P-gp, and therefore whether or not it is a sedating or non-sedating H₁-antagonist in accordance with the present invention. Accordingly, an H₁-antagonist candidate is non-sedating when it is determined to be a substrate for P-gp derived from humans, monkeys, mice, rats, dogs or any other species of mammal. This aspect of the present invention is based on the high degree of homology to be found among P-gp's from different mammal species, which in turn is a reflection of the highly conserved nature of the genetic material involved. P-gp from any mammalian source may, consequently, be used to determine whether a particular H₁-antagonist candidate is a substrate for P-gp in general, using methods well known in the art. As a practical matter, on the other hand, it has been found most convenient, and therefore preferred embodiments of the present invention comprise, methods of determination that utilize P-gp derived from humans and mice. MDR1 expressed P-gp, or mdr1a or mdr1a/1b expressed P-gp is the material of choice in the preferred methods of the present invention for determining whether an H₁-antagonist candidate is non-sedating or not. P-gp from these particular sources is readily available in pure form and is dependably uniform in character.

The fact that cetirizine is a P-gp substrate has been further substantiated by test results obtained from a study involving the administration of hydroxyzine, the metabolic precursor of cetirizine. From these test results it was also determined that the formation of cetirizine from hydroxyzine did not differ with regard to whether the mouse strain was KO or WT. This conclusion was based on the similarity of systemic exposure to cetirizine post-hydroxyzine administration in both strains. On the other hand, significant levels of cetirizine were measured in the brains of the KO mice, but not in the brains of the WT mice.

In a similar fashion, test results from a study involving formation of the active metabolite desloratadine after dosing of loratadine, were found to be comparable in plasma AUC between KO mice and WT mice, although at the same time no detectable brain tissue concentration of desloratadine after dosing loratadine was found in the WT or mdr1a/b KO mice. It has been concluded that this result was due to the low plasma concentration of desloratadine that was found to exist, and thus to the even lower brain tissue concentration of desioratadine that would follow, which was probably below the limit of quantification of the assay employed, i.e., 1.0 ng/mL.

By contrast, test results obtained from evaluations similar to the above-described studies have shown that sedating H₁-antagonist do not exhibit a significant difference in brain tissue concentration-over-time profiles in WT vs. KO mice. The AUC ratio of brain tissue to plasma of approximately 1 obtained for diphenhydramine, triprolidine, and hydroxyzine has led to the conclusion that they are not P-gp substrates.

In accordance with the present invention, it has been shown as described in detail herein, that the formation of cetirizine from hydroxyzine or desloratadine from loratadine is essentially the same in the mdr1a/1b KO and WT strains of mice. Accordingly, the genetically altered mdr1a/1b KO mice utilized in the method of the present invention are sufficiently identical to their genetically competent counterparts to assure that the conclusions drawn therefrom are accurate and reliable.

The description herein demonstrates that nonsedating H₁-antagonist are P-gp substrates, while sedating H₁-antagonist are not. Based on this important difference, the method of the present invention permits the reliable selection of nonsedating antagonists. The method of the present invention may be carried out using more than one procedure, although some procedures are preferred to others. For example, due to the low throughput of in vivo procedures, and with a view toward possible species-differences between animal and human P-gp, in vitro procedures using MDR1 transfected cell lines such as MDCK cells are preferred, and also offer the advantage of high throughput screening. See Soldner A, Benet L Z, Mutschler E and Christians U (2000), “Active transport of the angiotensin-II antagonist losartan and its main metabolite EXP 3174 across MDCK-MDR1 and caco-2 cell monolayers,” British J Pharmacol 129:1235-43; Pastan I, Gottesman M M, Ueda K, Lovelace E, Rutherford A V and Willingham M C (1988), “A retrovirus carrying an MDR1 cDNA confers multidrug resistance and polarized expression of P-gp in MDCK cells,” Proc Natl A cad Sci USA 85: 4486-90; and Horio, M, Chin K V, Currier S J, Goldenberg -S, Williams C, Pastan I, Gottesman M M and Handler J (1989), “Transepithelial transport of drugs by the multidrug transporter in cultured Madin-Darby canine kidney cell epithelia,” J Biol Chem 264: 14880-4.

Since the method of the present invention is one for selecting nonsedating H₁-antagonists, the compound that is selected will, accordingly, be a histamine H₁-antagonist and will also be nonsedating. The term “nonsedating” as used herein is intended to mean primarily, with reference to the pharmacologic properties of an agent, not producing a sedative effect, i.e., not allaying activity and excitement in a patient, not acting to calm said patient. The meaning of the term “nonsedating” as used herein with reference to the pharmacologic properties of an agent, also includes not producing somnolence, i.e., sleepiness or an unnatural drowsiness in a patient. The meaning of the term “nonsedating” as used herein with reference to the pharmacologic properties of an agent, is intended to further broadly include the absence of any significant tendency of said agent to affect adversely cognition, psychomotor performance, or mood. Adverse affects on, e.g., psychomotor performance, may be used as an additional, or even alternative, way of measuring the type and extent of any sedation that may be produced in a patient by a test agent that has been administered.

In addition to being nonsedating, a compound that is selected in accordance with the method of the present invention will also be a histamine H₁-antagonist. Methods for determining histamine H₁-antagonism are well known in the art and the medicinal chemist or biologist of ordinary skill in said art would be aware of a number of suitable choices of assays and procedures whereby such histamine H₁-antagonism might be readily and accurately determined. Thus, in order to carry out the method of the present invention, it is first necessary to establish a group of candidate compounds that have been determined to be H₁-antagonists. Once a determination of the existence or absence of histamine H₁-antagonism has been made and the group of candidate compounds thereby established, the next step of the method of the present invention comprises a determination of whether or not said H₁-antagonist candidates are substrates for P-gp. Preferred embodiments of this step of the method of the present invention, comprise a determination of the blood vs. brain distribution of each said candidate compound in mdr1a/1b KO mice vs. WT mice, or comprise a determination of bi-directional transport of said H₁-antagonist candidates in MDR1-MDCK cells. From calculations of these results it is possible to determine whether said H₁-antagonists are substrates for P-gp.

The principles that underlie the method of the present invention may be demonstrated by the following study which was carried out. Three sedating histamine H₁-antagonists, hydroxyzine, diphenhydramine, and triprolidine, were used in the study; and three nonsedating H₁-antagonists, cetirizine, loratadine, and desloratadine, were used in the study. The blood vs. brain distribution of all of these H₁-antagonists was examined in mdr1a/1b KO and WT mice after intravenous administration thereof. The results from the study demonstrated that the sedating H₁-antagonists hydroxyzine, diphenhydramine, and triprolidine are not P-gp substrates and have significantly greater brain penetration than the nonsedating H₁-antagonists. In contrast, the nonsedating H₁-antagonists cetirizine, loratadine, and desloratadine are P-gp substrates and exhibit reduced brain exposure as compared to the sedating H₁-antagonists. In addition, two sedating (triprolidine and diphenhydramine) and two non-sedating (cetirizine and desloratadine) H₁-antagonists were tested in MDR1-MDCK cells and the results showed that the two sedating H₁-antagonists were not P-gp substrates and the two non-sedating H₁-antagonists were P-gp substrates. Accordingly, a combined screening for H₁-receptor and P-gp affinities is used in the method of the present invention to select H₁-antagonists with a desired high level of potency, but without any concomitant, significant sedating effect.

### 5.0 DESCRIPTION OF PREFERRED EMBODIMENTS

There follows a description of working examples showing various aspects of carrying out the method of the present invention. These examples are intended to further illustrate the present invention and provide further guidance in accordance with which the method of the present invention may be more readily carried out by the artisan. The artisan will be aware of many other suitable variations of the method of the present invention that would suggest themselves to the artisan and which are available in the art.

The description which follows is for the purpose of illustrating the present invention and is not intended to in any way create limitations, express or implied, upon the scope of the present invention. The claims appended hereto are for the purpose of reciting the present invention, of expressing the contemplated scope thereof, and of pointing out particulars thereof.

### EXAMPLE

Hydroxyzine, triprolidine, and diphenhydramine utilized in this investigation were obtained from Sigma Chemical Co. (St. Louis, Mo.). Cetirizine, loratadine, and desloratadine utilized in this investigation were synthesized in accordance with procedures well known in the art to a purity of >99%. All the other chemicals and reagents were the highest grade available from commercial sources.

The animals used in this investigation comprised male FVB (control) and mdr1a/1b KO mice, 4-5 weeks of age (Taconic, Germantown, N.Y.), which were housed in separarte groups of 2 and 20, respectively, with free access to food and water, and maintained on a 12-hour light/dark cycle.

Blood-brain distribution of H₁-antagonists in WT mice and mdr1a/1b KO mice—The mice used in the study were administered each test compound individually, by intravenous injection at 5 mg/kg through the tail vein (<100 μL in less than 30 sec). Blood and brain samples were harvested at 2, 5, 15, 45, 120, 240, 480, and 1440 minutes post dose (n=3 mice per group). Plasma samples were obtained by centrifuging the blood samples at 13,000 rpm for 2 minutes. Brain tissue samples were rinsed with saline and blotted dry and weighed. Samples were stored at −20° C. before analysis by LC-MS.

Quantification of H₁-antagonists in plasma and brain samples: pretreatment—Loratadine and triprolidine plasma samples were extracted using liquid-liquid extraction. Briefly, to 100 μL of sample was added 10 μL of internal standard (i.s.). The compound of Formula (1.0.0) below and diphenhydramine were used as i.s. for loratadine and triprolidine, respectively. The compound of Formula (1.0.0) has the following structure:

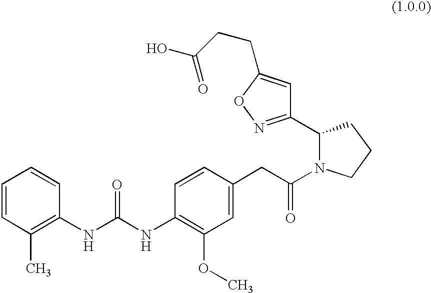

The samples were extracted by adding methyl tert-butyl ether (MTBE) (500 μL) using a Personal-550 Pipettor 96 Channels (Apricot Designs Inc., Monrovia, Calif.). An aliquot (350 μL) of the supernatant was transferred to a new set of tubes after mixing and centrifugation (3000 rpm×15 min). The supernatant was evaporated to dryness with Evaporex 96 Channels (Apricot Designs Inc., Monrovia, Calif.) under nitrogen gas, and the residue was reconstituted with mobile phase (acetonitrile: water containing 0.1% acetic acid, 50:50, v/v, 100 μL). Cetirizine, diphenhydramine, hydroxyzine and desloratadine were extracted via acetonitrile precipitation.

Briefly, to 100 μL of sample was added 300 μL of acetonitrile containing i.s. The compound of Formula (1.0.0) above was used as i.s. for diphenhydramine; loratadine was used as i.s. for hydroxyzine and desloratadine); and the compound of Formula (2.0.0) below was used as i.s. for cetirizine. The compound of Formula (2.0.0) has the following structure:

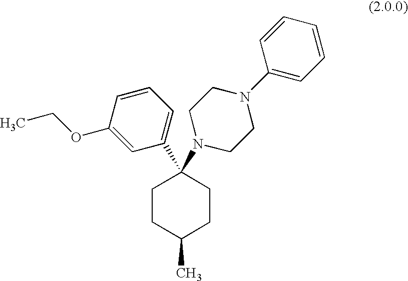

The remainder of the procedures were the same as for the liquid-liquid extraction. The brain tissue samples were homogenized after adding 4-volumes of saline. The resulting brain tissue homogenate (100 μL) was used for analysis. The method for extraction of brain tissue samples was identical to that described for the plasma samples using acetonitrile precipitation.

LC-MS-MS instrumentation and conditions—HPLC-MS consisted of a Hewlett-Packard (HP) 1100 quaternary pump with membrane degasser (Hewlett Packard, Palo Alto, Calif.), a Gilson 215 liquid handler (Gilson Inc., Middleton, Wis.) and a PE Sciex API 3000 mass spectrometer with a turbo ion spray interface (PE-Sciex, Thornhill, Ontario, Canada). An aliquot of the reconstitute (20 μL) was injected into an Asahipak ODP C18 column (2.6 i.d.×20 mm, Keystone Scientific, Inc., Charlotte, N.C.). The analytes and i.s. were eluted with a mobile phase composed of solvents A (95% water: 5% acetonitrile, containing 0.01% acetic acid) and B (5% water: 95% acetonitrile, containing 0.01% acetic acid) under the following gradient: 0-0.20 minutes, 100% of solvent A, 0.20-0.30 minutes, linear gradient from 1.00% solvent A to 100% solvent B, and then maintained at 100% of solvent B for up to 1.5 minutes. The analyte and i.s. were monitored based on the ion pair (parent and daughter) that is specific to each compound at a collision energy of positive 40 volts. The ion pairs (parent and daughter) under the current MS conditions for diphenhydramine, hydroxyzine, triprolidine, cetirizine, loratadine and desloratadine were 255.3/136.1, 374.9/202.2, 278.4/208.2, 388.9/202.2, 383.0/338.1, and 310.8/259.2, respectively. The linear range of quantification was from 1 ng/mL to 10 μg/mL. The peak areas of the analyte and i.s. were obtained using a MacQuan mass spectrometer (PE-Sciex, Thornhill, Ontario, Canada).

Estimation of pharmacokinetic parameters—Non-compartmental analysis was used to estimate the pharmacokinetic parameters such as AUC, systemic clearance (CL), volume of distribution (V_{d}) and half-life (t_{½}) for plasma concentration-time data. Only AUC was calculated for brain concentration-time data for each test compound.

Metabolism of H₁-antagonists in vivo—The major circulating metabolites of loratadine and hydroxyzine in mice were identified using LC-MS-MS with the aid of standards for said metabolites. The plasma samples were pooled and were pretreated in the same way as for quantification described above.

Transepithelial transport of two sedating H₁-antagonists, triprolidine and diphenhydramine, and two non-sedating H₁-antagonists, cetirizine and desloratadine, in MDR1-MDCK cells—Cells were obtained from the laboratory of Piet Borst, The Netherlands Cancer Institute, Amsterdam, The Netherlands, were seeded at a density of 2×10⁵ cell/ml on 96-well plates. Cells were incubated in a standard growth media at 37° C. with 95% humidity and 5% CO₂ and maintained as described in the literature, e.g., Lala P, Ito S, and Lingwood C (2000), “Retrovial transfection of Madin-Darby Canine Kidney cells with human MDR1 results in a major increase in globotriaosylceramide and 10⁵- to 10⁶-fold increased cell sensitivity to verocytotoxin—Role of P-glycoprotein in glycolipid synthesis,” J Biol Chem 275: 6246-6251. For measurement of transepithelial transport of H₁-antagonists, triprolidine, diphenhydramine, cetirizine and desloratadine was added to the donor side (either apical, A or basal, B) at a final concentration of 2 μM to initiate the transport study. The appearance of these H1-antagonists at the receiver side was determined by sampling from the receiver side at 5 hr post initiation of the transport study. The concentrations were determined by LC-MS as described previously. The apparent permeability (Pₐₚₚ) was calculated according to the following equation:

$$Papp ( cm \text{/} sec ) = \frac{Concentration , Receiver * V , Receiver}{Td * Area * Concentration , Donor}$$

where “Concentration, Receiver” and “Concentration, Donor” refer to concentration of the test H₁-antagonist at the receiver and donor side, respectively. “V, Receiver” refers to volume of solution at the apical (0.075 ml) or basal (0.250 ml) side. “Td” is the duration of transport conducted, and “Area” refers to the surface area available for transport between donor side and receiver side, which is 0.064 cm². The Pₐₚₚ values were calculated for both A-to-B and B-to-A transport. The ratio of Pₐₚₚ values between B-to-A and A-to-B was used as the efflux index. A ratio value which is equal to or larger than 2 is considered to show efflux mediated by MDR1.

RESULTS obtained following the procedures described above are set forth in detail in the paragraphs that follow:

Blood-brain disposition of six H₁-antaqonists in WT and KO mice—In all cases, the plasma concentration-time profiles were superimposable between the two strains of mice. Consequently, no difference in systemic pharmacokinetic (PK) parameters between the two strains of mice was observed for the H₁-antagonists tested in the investigation described herein. The results obtained are set forth in the following table of values identified as TABLE 1:

TABLE 1
PK Parameters of Six H₁-Antagonists
in mdr1a/1b KO Mice and WT (FVB) Mice

| | PK PARAMETERS | | | | | |
|---|---|---|---|---|---|---|
| | CL (mL/min/kg) | | V_{d} (L/kg) | | t_{1/2} (hr) | |
| H₁-ANTAGONISTS | KO¹ | WT² | KO | WT | KO | WT |
| Cetirizine | 5.4 | 7.5 | 1.1 | 0.9 | 7.8 | 3.6 |
| Loratadine | 83 | 99 | 2.1 | 2.2 | 0.5 | 0.4 |
| Desloratadine | 20 | 23 | 3.2 | 3.0 | 3.2 | 2.8 |
| Hydroxyzine | 70 | 80 | 2.4 | 3.7 | 2.2 | 1.7 |
| Diphenhydramine | 62 | 66 | 1.6 | 1.3 | 0.8 | 0.6 |
| Triprolidine | 134 | 119 | 2.3 | 2.0 | 0.5 | 0.3 |

¹KO = knockout.
²WT = wild type.

The CL, V_{d} and t_{½} values, as set forth in TABLE 1 above, were found to be comparable between KO and WT mice for all six H₁-antagonists. Within the six H1-antagonists, cetirizine had the smallest V_{d} (1 L/kg) followed by diphenhydramine (1.3-1.6 L/kg), loratadine and triprolidine (˜2 L/kg), and hydroxyzine (2.4-3.7 L/kg). Likewise; cetirizine was found to have the lowest C1 (5.4-7.5 mL/min/kg), followed by desloratadine (20-23 mL/min/kg), diphenhydramine (62-66 mL/min/kg), hydroxyzine (70-80 mL/min/kg), loratadine (83-99 mL/min/kg) and triprolidine (119-134 mL/min/kg).

A 4-fold higher brain tissue AUC was found in KO mice than was found in WT mice for cetirizine, while the Cₘₐₓ in brain tissue was 0.09 μg/mL and 0.28 μg/mL for WT and KO mice, respectively. After dosing hydroxyzine, no difference in hydroxyzine brain tissue concentration-time profile between WT and KO mice was observed. Hydroxyzine peaked at 2 minutes in brain tissue for both WT and KO mice post i.v. administration. The brain-to-plasma AUC ratio was observed to be 3.8 and 4.8 for wild WT and KO mice, respectively. The KO mice showed slightly higher plasma AUC than the WT mice (418 vs. 287 μg/mL×min). In contrast, cetirizine was detected in KO mouse brain tissue after hydroxyzine administration with an AUC of 67 μg/mL×min, whereas cetirizine brain tissue concentration in WT mice was below the limit of quantification of the assay that was used, i.e., 10 ng/mL.

Similar to cetirizine, loratadine also had a higher brain-to-plasma AUC ratio in KO mice as compared to the WT mice (2.7 vs. 1.6). The brain concentration of loratadine peaked at 2 minutes post dose and had a Cₘₐₓ of 14.7 μg/mL and 6.9 μg/mL for KO mice and WT mice, respectively. It was also observed that loratadine cleared from the brain tissue very efficiently, with more rapid clearance from brain tissue in WT mice than in KO mice. Desloratadine, the major active metabolite of loratadine, was found to have a plasma concentration-time profile after dosing of loratadine such that no significant difference was observed in the formation of desloratadine in the two strains of mice. The AUC was 25 μg/mL×min and 30 μg/mL×min for WT mice and for KO mice, respectively. No detectable brain tissue concentration of desloratadine was observed post loratadine administration in either strain of mice. When desloratadine was dosed directly to the two strains of mice, no brain tissue concentration was detected in the WT mice except at 5 minutes post dose, whereas a high level of desloratadine was found in the KO mice. The brain AUC of desloratadine in KO mice was 3548 μg/mL×min.

Diphenhydramine, another typical sedating H₁-antagonist, was found to have great brain tissue penetration in both strains of mice. Peak brain tissue concentration was 23 μg/mL and 16 μg/mL at 2 minutes post dose in KO mice and WT mice, respectively; while no significant difference in brain tissue AUC was observed between WT mice (686 μg/mL×min) and KO mice (754 μg/mL×min). In a similar fashion, triprolidine was observed to have good brain tissue penetration, as reflected in the brain-to-plasma AUC ratio, which was 6.2 and 3.5 for WT mice and KO mice, respectively. The brain tissue concentration was observed to peak at 2 minutes.

Metabolism of H₁-antagonists in vivo—The major metabolite for hydroxyzine and loratadine were determined to be cetirizine and desloratadine, respectively. This determination was confirmed with LC-MS-MS fragmentation of each metabolite as compared to that of the corresponding standard.

The above-discussed results of the comparison of brain-to-plasma partition in sedating and nonsedating H₁-antagonists are set out in TABLE 2 below:

TABLE 2
Comparison of Brain-to-Plasma Partition of Sedating and Nonsedating H₁-Antagonists

| COMPOUND | STRUCTURE | KO/WT¹ | P-GP² | SEDATING³ |
|---|---|---|---|---|
| Cetirizine | 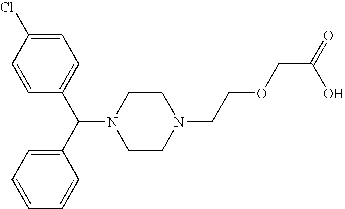 | 4.4 | Yes | No |
| Loratadine | 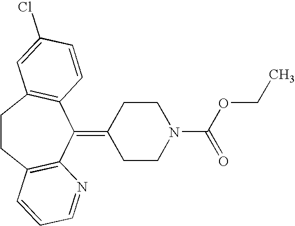 | 1.6 | Yes | No |
| Desloratadine | 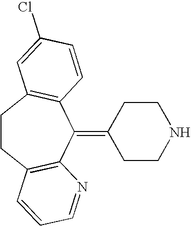 | >14 | Yes | No |
| Hydroxyzine | 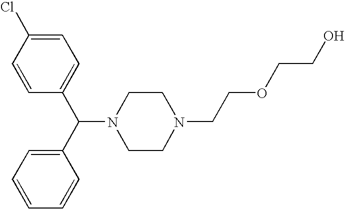 | 1.2 | No | Yes |
| Diphenhydramine | 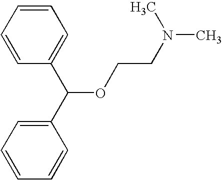 | 1.0 | No | Yes |
| Triprolidine | 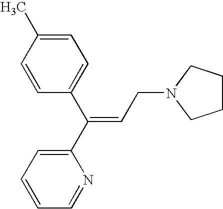 | 0.6 | No | Yes |

¹Ratio of brain-to-plasma AUC ratios between KO and WT mice.
²Whether or not the test compound is a substrate for P-gp.
³Whether or not the test compound is sedating.

Transepithelial transport of two sedating H₁-antagonists, triprolidine and diphenhydramine, and two non-sedating H₁-antagonists, cetirizine and desloratadine, in MDR1-MDCK cells—Result is shown in Table 3. Both cetirizine and desloratadine showed asymmetric permeability where B-to-A permeability was significantly higher than the opposite direction, indicating MDR1-mediated transport of cetirizine and desloratadine. Comparable A-to-B and B-to-A permeability was observed for triprolidine and diphenhydramine, suggesting that these two sedating H₁-antagonists are not MDR1 substrates. The results from MDR1-MDCK cells were consistent with results from the mdr1a/b KO mouse studies, suggesting that non-sedating H₁-antagonists are substrates for both murine and human P-gp and that sedating H₁-antagonists are not substrates for either murine or human P-gp. This observation is consistent with results obtained for fexofenadine. See Cvetkovic et al., ibid.

TABLE 3
Transepithelial Transport of Sedating/Nonsedating
H₁-Antagonists in MDR1-MDCK Cells

| | Papp (cm/sec) × 10⁻⁶ | | | |
|---|---|---|---|---|
| H₁-ANTAGONISTS | A→B | B→A | B→A/A→B | MDR1 Substrate? |
| Triprolidine | 25.9 | 24.5 | 0.9 | No |
| Diphenhydramine | 24.8 | 21.0 | 0.9 | No |
| Cetirizine | 2.6 | 15.2 | 5.8 | Yes |
| Desloratadine | 1.8 | 16.3 | 9.1 | Yes |

What is claimed is:

1. A method for selecting a nonsedating H₁-antagonist comprising determining whether a candidate H₁-antagonist is a substrate for P-gp.

2. A method according to claim 1 wherein said P-gp is expressed by MDR1 or by mdr1a/1b.

3. A method according to claim 2 for selecting a nonsedating H₁-antagonist by ascertaining whether a candidate H₁-antagonist is a substrate for P-gp expressed by mdr1a/1b, comprising the step of determining the brain-to-plasma concentration ratio in mdr1a KO mice or mdr1a/1b KO mice, and in WT mice, and selecting as said nonsedating H₁-antagonist said candidate in the event that the brain-to-plasma concentration ratio for said KO mice to the brain-to-plasma concentration ratio for said WT mice is 1.5 or greater.

4. A method according to claim 3 wherein said brain-to-plasma concentration ratio for said KO mice to the brain-to-plasma concentration ratio for said WT mice is 3.0 or greater.

5. A method according to claim 3 wherein said brain-to-plasma concentration ratio for said KO mice to the brain-to-plasma concentration ratio for said WT mice is 5.0 or greater.

6. A method according to claim 3 wherein said brain-to-plasma concentration ratio for said KO mice to the brain-to-plasma concentration ratio for said WT mice is 10.0 or greater.

7. A method according to claim 3 wherein said brain-to-plasma concentration ratio for said KO mice to the brain-to-plasma concentration ratio for said WT mice is 15.0 or greater.

8. A method according to claim 2 for selecting a nonsedating H₁-antagonist by ascertaining whether a candidate H₁-antagonist is a substrate for P-gp expressed by mdr1a/1b, comprising the step of determining the brain-to-plasma AUC value in mdr1a KO mice or mdr1a/1b KO mice, and in WT mice, and selecting as said nonsedating H₁-antagonist said candidate in the event that the ratio of AUC values for said KO mice to said WT mice is 1.5 or greater.

9. A method according to claim 1 for selecting a non-sedating H₁-antagonist by ascertaining whether an H₁-antagonist candidate is a substrate for P-gp comprising (1) the step of making a determination that is carried out in vitro in a P-gp over-expressed cell line vs. the appropriate control cell line; (2) the step of making a determination that is carried out in situ in a P-gp competent animal in the presence vs. the absence of a P-gp modulator, or in situ in a P-gp KO or compromised animal vs. its P-gp competent counterpart; or (3) the step of making a determination that is carried out in vivo in a P-gp competent animal in the presence vs. the absence of a P-gp modulator, or in vivo in a P-gp KO or compromised animal vs. its P-gp competent counterpart.

10. A method according to claim 9 wherein said P-gp is over-expressed by MDR1 or mdr1a or mdr1a/b in vitro by a cell line.

11. A method according to claim 10 wherein said cell line comprises CHO cells, Caco-2 cells, MDCK cells, KB 8-5 cells, NIH 3T3 cells, drug-resistant tumor cells, or LLC-PK1 cells.

12. A method according to claim 9 carried out in situ in a P-gp competent animal by organ perfusion in the presence vs. absence of a P-gp modulator.

13. A method according to claim 9 carried out in situ in a P-gp KO or compromised animal vs. a P-gp competent animal by organ perfusion.

14. A method according to claim 12 carried out by intestinal perfusion.

15. A method according to claim 12 carried out by brain perfusion.

16. A method according to claim 12 carried out by liver perfusion.

17. A method according to claim 1 for determining whether an H₁-antagonist candidate is a substrate for P-gp in vivo in a P-gp competent animal, comprising the step of comparing PK parameter values in the presence vs. absence of a P-gp modulator.

18. A method according to claim 1 for determining whether an H₁-antagonist candidate is a substrate for P-gp in vivo in a P-gp KO or compromised animal vs. P-gp competent counterpart, comprising the step of comparing PK parameter values between the two strains of animals.

19. A method according to claim 10 wherein said determination of whether a H₁-antagonist candidate is a substrate for P-gp is based on the ratio of permeability from basal-to-apical vs. apical-to-basal.

20. A method according to claim 12 wherein said determination of whether a H₁-antagonist candidate is a substrate for P-gp is based on the difference in uptake of said candidate H₁-antagonist in said organ.

21. A method according to claim 13 wherein said determination of whether a H₁-antagonist candidate is a substrate for P-gp is based on the difference in uptake of said candidate H₁-antagonist in said organ.
